(19) United States
(12) Patent Application Publication
(10) Pub. No.: US 2021/0106083 A1
(43) Pub. Date: Apr. 15, 2021

(54) FRONT-OPENING HOSPITAL GOWN WITH RADIOLUCENT SNAPS

(71) Applicant: Holly Spiegle, Williams, IN (US)

(72) Inventor: Holly Spiegle, Williams, IN (US)

(21) Appl. No.: 16/882,370

(22) Filed: May 22, 2020

Related U.S. Application Data
(60) Provisional application No. 62/852,152, filed on May 23, 2019.

(51) Int. Cl. A41D 13/12; C08K 7/06; C08K 3/04; G01R 33/28

(52) U.S. Cl. CPC A41D 13/1236; C08K 7/06; A61B 5/055; G01R 33/28; A41D 2300/324; C08K 3/04

(57) ABSTRACT

A radiolucent gown including fabric formed into an open anterior panel, a posterior panel and sleeves. Radiolucent anterior snaps and radiolucent sleeve snaps secure the anterior panel and the sleeves. The snaps are all manufactured from a composite material having a 10-45% chopped carbon fiber content and the carbon fibers have an average aspect ratio (L/W) of between 50-120. The snaps have a radio density of less than 60 Hounsfield units, and preferably less than 35 Hounsfield units at a tube voltage of 100-120 kVp. In one embodiment, the radio density of all snaps is between 35-60 Hounsfield units. The snaps all have a radio density of less than 35 Hounsfield units at a tube voltage of 100-120 kVp. Preferably, the snaps are the same shape and size and are size 20 snaps, having a 1.2 inch diameter.

### FIELD OF THE INVENTION

The present invention is a gown, particularly a gown worn during medical treatment and diagnostic procedures.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

Hospital gowns are used by patients in a medical facility such as a hospital. Specialized gowns are used for particular medical diagnostics procedures, medical assessments, and medical interventions such as wound care. Most gowns open from the posterior of patient and have either posterior or lateral snaps for holding the gown in place.

An anterior opening gowns makes frontal access to the patient easier. Anterior opening gowns, however, are not typically used. This may be due in part to the fact that anterior snaps may interfere with X-ray diagnostic work.

Many hospitals have various departments including radiology, magnetic imaging, coronary care, general surgery, oncology, and many others. Each department may have particular requirements for gown design and functionality. For example, the magnetic imaging department may require gowns with non-metallic components, whereas the radiology department may require radiolucent gowns. An intensive care department may require gowns with a pocket for securing monitoring equipment. It is common for patients to change gowns for short visits with each department of a hospital. Changing gowns frequently increases laundry costs, takes staff time, and inconveniences patients.

X-ray imaging typically relies upon radiation having a wavelength in the range of ten picometers to ten nanometers. This radiation, in traditional usage, penetrates radiolucent soft tissue and reflects from radio-dense structures, including bones. Modern usage includes utilizing a radio-dense contrast material or media which is passed through the blood stream, gastrointestinal tract, spinal fluid, or other fluid and is utilized to highlight computed tomography (CT) or X-ray images. Examples of radio-dense contrast materials include titanium, tungsten, barium sulfate, and zirconium oxide.

Magnetic resonance imaging, or MRI, is a way of obtaining detailed images of organs and tissues throughout the body without the need for x-rays or “ionizing” radiation. Instead, MRI uses a powerful magnetic field, radio waves, rapidly changing magnetic fields, and a computer to create images that show whether or not there is an injury, disease process, or abnormal condition present.

An electrocardiogram (ECG) typically monitors heart rate and typically relies on wired electrodes attached to a patient's skin. Some of these electrodes are radiolucent such as those marketed by Cardinal Health, Inc. as Kendall™ 930 series foam electrodes. Many medical patients need continuous monitoring and the benefit of radiolucent electrodes is that these can remain in place when a patient also needs diagnostic work performed with an MRI procedure, for example.

Computer axial tomography (CAT) uses multiple x-ray projections taken from different angles to produces a 3D image that is capable of determining the contrast of density of softer tissues.

Radiolucence is the opposite of radiopacity, both of which are aspects of radiodensity. Radiolucent materials allow X-rays to pass freely. Radiopaque materials absorb, refract or reflect X-rays. ASTM F640 “Standard Test Methods for Determining Radiopacity for Medical Use” is a useful standard for articulating radiodensity, or expressing the degree of radiolucence. In practice, radiolucent structures allow passage of X-rays and show up a nearly black (virtually undetectable) on traditional X-ray images. Radiolucency (radiolucense) as used herein is defined as a material having less than 30 Hounsfield units (HU) per gram of material with a tube voltage of 110 kVp.

The Hounsfield scale is a quantitative scale for describing computed tomography (CT) density. The Hounsfield scale provides one of many quantitative scales to articulate the concept of radiodensity, and thus radiolucence. Water has a HU density of zero. Air has a HU value of −1000 HU. Radiopaque, or dense materials, such as bone have a HU density value on the order of +1000, for example.

Iodinated and other types of contrast media used in conjunction with medical diagnostic procedures, such as contrast CT imaging, have a relatively low radiodensity of, for example, 25-30 Hounsfield units per milligram of iodine per milliliter at a tube voltage of 100-120 kVp.

Examples of biological materials and their HU values are expressed in Table 1.

TABLE 1

| Hounsfield units (HU) | Tissue |
|---|---|
| 1000 | Bone, calcium, metal |
| 100 to 600 | Iodinated CT contrast |
| 30 to 500 | Punctate calcifications |
| 60 to 100 | Intracranial hemorrhage |
| 35 | Gray matter |
| 25 | White matter |
| 20 to 40 | Muscle, soft tissue |
| 0 | Water |
| −30 to −70 | Fat |
| −1000 | Air |

Various Electrocardiogram (ECG) devices are equipped with special electrodes that attach to the body and are wired to ECG monitoring equipment and computer systems. During a Magnetic Resonance Imaging diagnostic procedure, the ECG electrodes and wires may need to be attached to the patient. The magnetic fields of the MRI machine may cause metallic objects to heat and could burn a patient. Also, magnetic fields induced by the MRI machine in some ferrous and magnetically responsive objects may cause anomalies, artifacts, and induced magnetic fields around metallic objects including some ECG electrodes.

As a result various ECG electrodes have been adapted and evaluated to be compatible with MRI equipment. Some can be used with static magnetic fields of 1.3-3 Tesla in strength. Others are being adapted for use in stronger and variable magnetic fields.

Patients seldom wish to enter a MRI machine unclothed. Some gowns, however, have metallic snaps, or may have components impregnated with metals to inhibit microbial contamination. Even gowns without metallic components can be problematic because the wires from an ECG electrode must pass from the body of the patient to the ECG monitoring equipment and a typical gown may be not be able to easily enable the wiring, while preserving the modesty of a patient. The use of x-ray machines generally requires radiolucent materials to be used. Thus, a person clothed on a non-radiolucent gown, may have to change to an x-ray safe gown.

What is desired is a MRI safe gown that X-ray safe and is usable under numerous medical assessments. What is also desired is a gown that is radiolucent and can be used with medical assessments ECG, X-ray, wound care and monitoring and diagnostic equipment while preserving the modesty of a patient. What is further desired is a gown that enables convenient access to a patient during medical procedures.

### SUMMARY OF THE INVENTION

The present invention includes a radiolucent gown adapted for selectively using imaging magnetic resonance imaging (MRI) scanner, and x-ray machine, or an electrocardiogram (ECG) device on a user during a diagnostic session.

The radiolucent gown includes fabric formed into an open anterior panel, a posterior panel and sleeves. Radiolucent anterior snaps and radiolucent sleeve snaps secure the anterior panel and the sleeves. The snaps are all manufactured from a composite material having a 10-45% chopped carbon fiber content and the carbon fibers have an average aspect ratio (L/W) of between 50-120. The snaps have a radio density of less than 60 Hounsfield units, and preferably less than 35 Hounsfield units at a tube voltage of 100-120 kVp. In one embodiment, the radio density of all snaps is between 35-60 Hounsfield units. The snaps all have a radio density of less than 35 Hounsfield units at a tube voltage of 100-120 kVp. Preferably, the snaps are the same shape and size and are size 20 snaps, having a 1.2 inch diameter.

It is noteworthy that the configuration of the gown with the various openings and arrangement of the snaps and panels cooperates with the radiolucent snaps to enable the gown to be used for x-rays because of the use of radiolucent materials, MRI machines due to the use of non-ferrous and non-magnetic materials, and with ECG machines because the materials are non-conductive of electricity.

The gown is front opening in case the patient needs CPR or other emergency procedure. The sleeves snap in a way that enables ease in use of an intravenous device. The pockets have openings for wires from an electrical device such as an ECG.

Accordingly, the configuration of the gown, including snap placement and material characteristics cooperate with each other to make a gown that is usable in numerous circumstances, which eliminates the need of patients to change gowns frequently. This eases patient discomfort, speeds the ability of health care workers to diagnose and treat the patient. Enables the patient to go from an x-ray to an MRI machine without changing. Further since the gowns need not be changed as frequently, this reduces the need for laundry which wears the gowns considerably. The gown achieves longer life through the ability to be used for more circumstances because laundry wear is lessened. This cooperates with the use of carbon fiber snaps that also lengthen the useful life of the gown.

The gown particularly includes an anterior panel having a length and two overlapping fabric sections join-able by a plurality of radiolucent anterior snaps that are alignable along a sagittal plane of the user when in use.

A posterior panel has a top defining a “v” shaped opening with a top tie integrated with the top of the “v” shaped opening on the top of the posterior panel, the “v” shaped opening having a center-line that is alignable along the sagittal plane of the user when in use. Two sleeves attach between the anterior (front) panel and the posterior (rear) panel and intersecting a coronal plane of the user when in use. A plurality of radiolucent sleeve snaps laterally aligned along each of the two sleeves and intersecting the coronal plane of the user when in use.

The sleeve snaps and the anterior snaps cooperate with the tie to enable selective and efficient access to the user during medical diagnosis and treatment without interfering with the magnetic resonance imaging scanner. Two pockets formed on each side of the sagittal plane of the anterior panel, the fabric sections of each anterior panel defines a hole for enabling a telemetry wire to pass through each anterior panel. Each pocket overlaps the respective hole to normally shield the hole from view, and to enable the pocket to be capable of loosely holding the telemetry wire.

A magnetic resonance imaging scan of the user may be simultaneously performed with a magnetic resonance imaging device while simultaneously and conveniently monitoring the user with the electrocardiogram equipment, or other electrical device that is wired to the user.

In one embodiment of the invention, the anterior panel defines an anterior panel “v” neck. A plurality of radiolucent anterior snaps is engaged during use. The “v” neck has an opening angle of between 45-65 degrees. In a variation of this embodiment, the “v” neck opening has an angle of approximately 55 degrees when the snaps are snapped.

The snaps, in an alternate embodiment are buttons. Preferably, the buttons or snaps are radiolucent so that they can be used in an x-ray machine safely. The snaps are non-conductive to electricity. The snaps are durable and resist solvents and laundry and sterilization chemicals. The snaps are also non-metallic and magnetically inert. With these requirements met, the gown in particular configurations can be used continuously by a patient during treatment or a hospital stay.

The gown preferably has a single side tie formed below the top tie to enable the posterior panel to overlap itself during use. The side tie connects one side of the posterior panel to the anterior panel.

The gown has a bottom edge and twelve anterior snaps, the first of which defines the base of the “v” neck opening angle of the anterior panel, the second of which is 6.3 inches below the first, the third is 8.2 inches below the second, the fourth is 8.2 inches below the third, the fourth is 8.2 inches below the third, the fifth is 8.8 inches below the fourth, the sixth is 8.2 inches below the fifth, the seventh is 8.2 inches below the sixth, the eighth is 7.9 inches below the seventh, the ninth is 7.9 inches below the eighth, the tenth is 8.2 inches below the ninth, the eleventh is 8.0 inches below the tenth, the twelfth is 7.9 inches below the eleventh, and the distance from the twelfth to the bottom edge of the gown is 3.8 inches, whereby the distance between the snaps decreases near the bottom edge to assure that the anterior panel will remain aligned when the user walks.

Each of the two sleeves has four sleeve snaps. Each of the sleeve snaps are size 20 snaps having a 1.2 inch diameter for ease of use by a user.

The sleeve snaps and the anterior snaps are formed from a cap, a socket and a stud fabricated from a non-metallic material resistant to microbial contamination.

The sleeve snaps and the anterior snaps are fabricated from a thermoplastic material selected from the group consisting of polypropylene, polyethylene, polyvinyl chloride, acrylonitrile butadiene styrene, and polycarbonate. The sleeve snaps are preferably reinforced with chopped carbon fiber.

In a variation of the invention, the sleeve snaps and the anterior snaps are powder coated to inhibit microbial contamination.

In yet another variation of the invention, the sleeve snaps are fabricated from injection molding and are impregnated with an anti-microbial compound.

A method of the present invention includes simultaneously using imaging magnetic resonance imaging scanner and an electrocardiogram device on a user wearing the gown of the present invention.

The method particularly includes providing a user with a gown having an anterior panel having a length and two overlapping fabric sections join-able by a plurality of non-metallic anterior snaps that are alignable along a sagittal plane of the user when in use; a posterior panel having a top defining a “v” shaped opening with a top tie integrated with the top of the “v” shaped opening on the top of the posterior panel, the “v” shaped opening having a center-line that is alignable along the sagittal plane of the user when in use; two sleeves attached between the front panel and the rear panel and intersecting a coronal plane of the user when in use; a plurality of non-metallic sleeve snaps laterally aligned along each of the two sleeves and intersecting the coronal plane of the user when in use; the sleeve snaps and the anterior snaps cooperate with the tie to enable selective and efficient access to the user during medical diagnosis and treatment without interfering with the magnetic resonance imaging scanner; two pockets formed on each side of the sagittal plane of the anterior panel, the fabric sections of each anterior panel defines a hole for enabling a telemetry wire to pass through each anterior panel, and each pocket overlaps the respective hole to normally shield the hole from view, and to enable the pocket to be capable of loosely holding the telemetry wire. The method further includes attaching an electrocardiogram electrode having a wire attached to the electrode and wired connection to electrocardiogram monitoring equipment to the user and threading the wire through the hole.

Next the method performs a magnetic resonance imaging scan of the user with the magnetic resonance imaging device.

### BRIEF DESCRIPTION OF THE DRAWINGS

FIG. 1 is a front view user wearing a gown in an MRI machine with an ECG attached to the user.

FIG. 2 is a rear view of the user wearing a gown.

FIG. 3 is a side view of a portion of the gown as seen along the line 2-2 of FIG. 2.

FIG. 4 is an upper rear view of the gown.

FIG. 5 is a snap kit.

### DETAILED DESCRIPTION

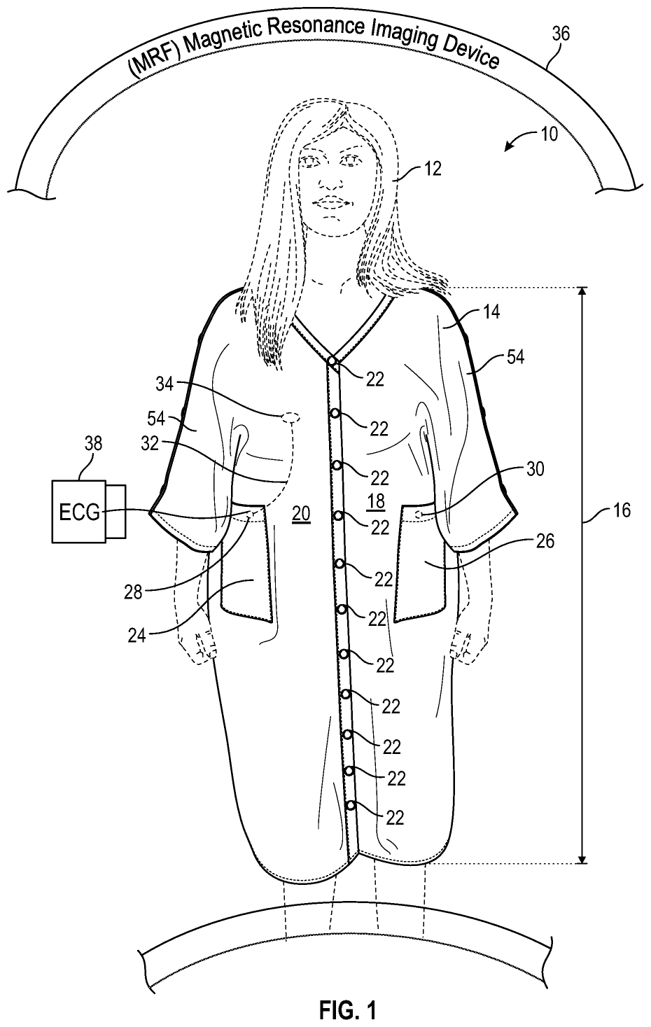

FIG. 1 shows the gown of the present invention, generally designated with the reference numeral 10. A user 12 wears the gown 10. The gown 10 has anterior panel 14 having length 16 and two overlapping fabric sections 18 and 20. The fabric sections 18 and 20 are join-able by a plurality of non-metallic anterior snaps 22 that are alignable along a sagittal plane of the user when in use.

Two pockets 24 and 26 are formed on each side of the sagittal plane of the anterior panel 14. The fabric sections 18 and 20 of the anterior panel 14 defines a hole 28 and 30, respectively for enabling a telemetry wire 32 to pass through the anterior panel 14. Each pocket 24 and 26 overlaps the respective hole 28 and 30 to normally shield the hole 24 and 30 from view, and to enable the pocket 24 and 26 to be capable of loosely holding the telemetry wire 32.

A magnetic resonance imaging device 36 surrounds the user 12. An electrocardiogram device 38 attaches via a wired connection through the wire 32 to at least one electrode 34 to the patient. It can be appreciated that various monitoring systems, including electrocardiogram devices are equipped with multiple electrodes. In one embodiment, there are five wired electrodes that attach to the shoulders, below each side of the rib cage, and near the sternum. The gown 10 of the present invention allows these wires to readily attach to the user without disrupting the gown 10 placement. This protects the modesty of the user. The shoulder electrodes can have wires passing through the top of the gown 10, those attaching below and near the rib cage can pass through the holes 28 and 30 with ease, and without requiring the unsnapping of the snaps 22.

The anterior panel 14 has two pockets 24 and 26 formed on each side of the sagittal plane of the anterior panel 14. The fabric sections of each side of the anterior panel 14 defines a hole 30 extending through each side of the anterior panel 14. The hole 30 enables a telemetry wire to pass through one side, or each side, of the anterior panel 40. Each pocket overlaps the respective hole to normally shield the hole from view, and to enable the pocket 24 and 26 to loosely hold the telemetry wire in a fixed position as the wire 32 extends through the hole. The pocket inhibits axial movement of the telemetry wire 32 to limit movement of the wire 32 that could dislodge or disable the electrode 34.

The user 12 has at least one electrocardiogram (ECG) electrode 34 attached to some part of the body of the user 12. The ECG electrode 34 has a wire 32 attached to the electrode 34 and wired connection to electrocardiogram monitoring equipment 38. The wire 32 threads through the hole 28 in the gown 10 and the electrocardiogram equipment 38 monitors the user 12. In an alternate embodiment, the ECG monitoring device 38 is replaced or supplemented with another diagnostic device such as a RR or a SpO₂ detector having a wired connection via the hole 28 to the user. The ECG can continue monitoring the user 12 while the user enters and maintains a position in the MRI device 36.

While a single electrode 34 is shown, various electrodes can be used, as well as other diagnostic leads, wires, intravenous delivery devices, and other therapeutic devices.

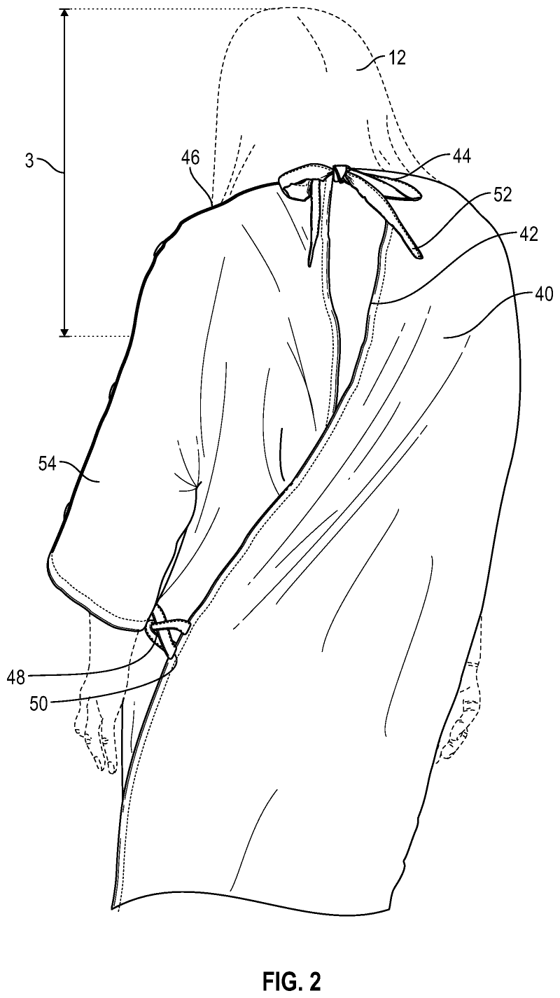

FIG. 2 shows the gown 10 having a posterior panel 40 having a top defining a “v” shaped opening 42 with a top tie 44 integrated with the top of the “v” shaped opening 42 on the top 46 of the posterior panel 40. The “v” shaped opening 42 has a center-line that is alignable along the sagittal plane of the user 12 when in use. As shown the posterior panel 40 has two overlapping sections that interconnect via the top tie 44, and a single lateral tie 48. Each tie 44 and 48 has two strips of cloth that each have a fixed end 50 attached to one section of the posterior panel 40 and a free end 52 that removably ties to the other strip of cloth to form the tie 44 and 48 respectively. The gown 10 has two sleeves 54.

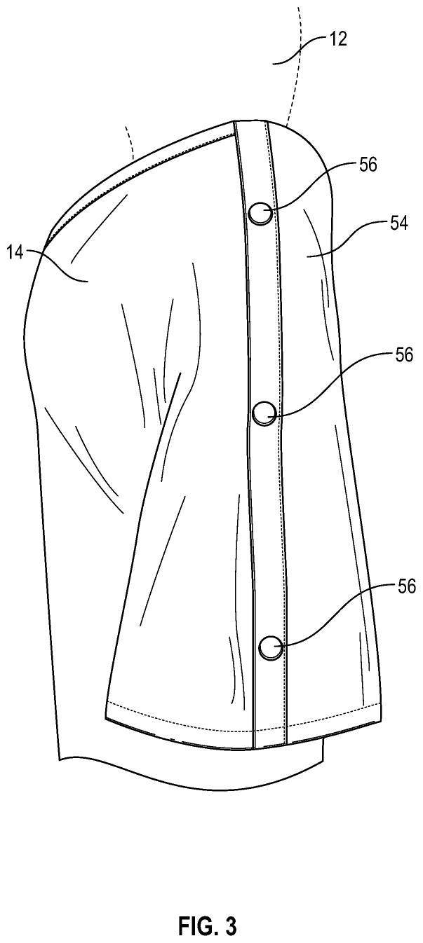

FIG. 3 shows a portion of the gown 10 worn by the user 12 as seen along the line 3-3 of FIG. 2. The gown 10 has a sleeve 54 attached between the anterior panel 14 and the posterior panel 40. The sleeve 54 aligns generally along the coronal plane of the user 12 when in use. A plurality of non-metallic sleeve snaps 56 laterally aligned along each of sleeve 54 and intersecting the coronal plane of the user when in use;

The sleeve snaps 56 and the anterior snaps 22 (FIG. 1) cooperate with the ties 44 and 48 to enable selective and efficient access to the user during medical diagnosis and treatment without interfering with the magnetic resonance imaging scanner. A wire, such as a telemetry wire 32 can therefore pass between the snaps 22, the snaps 56, the ties 44 and 48, as well as the overlapping fabric sections of the posterior panel 40. This enables the user 12 to lie comfortably in an Mill device 36 while establishing a wired connection to an ECG 38 or other diagnostic device, or to enable continuous use of an intravenous delivery system.

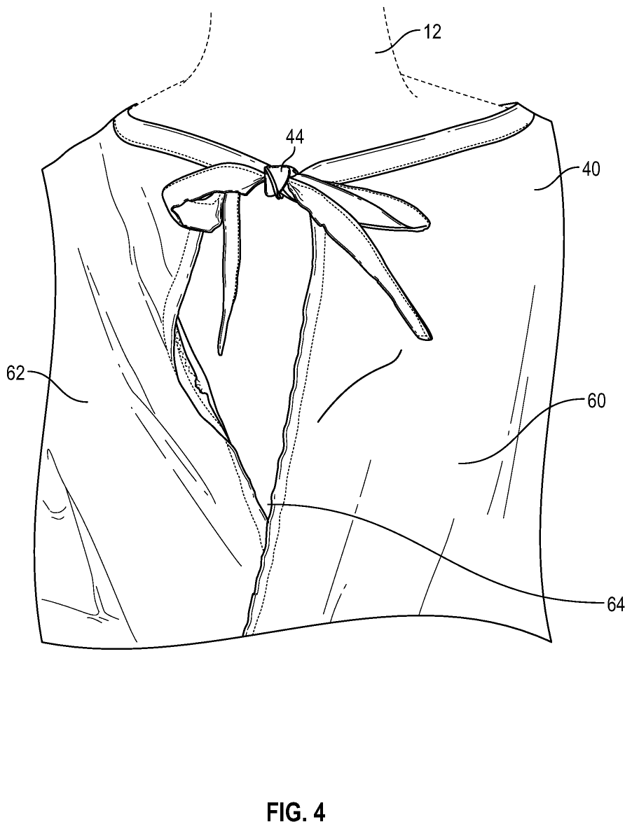

FIG. 4 shows the posterior panel 40 on the user 12. The posterior panel 40 has two overlapping sections 60 and 62. The tie 44 secures the top 46 of the posterior panel 40 near the neckline of the user 12. The tie 44 and the overlapping sections 60 and 60 define a “v” shaped opening to enable ready access to the users back. This enables wires to extend through the “v” shaped opening 64. The “v” shape inhibits bunching of the gown, creating an uncomfortable lump for a patient in a prone position.

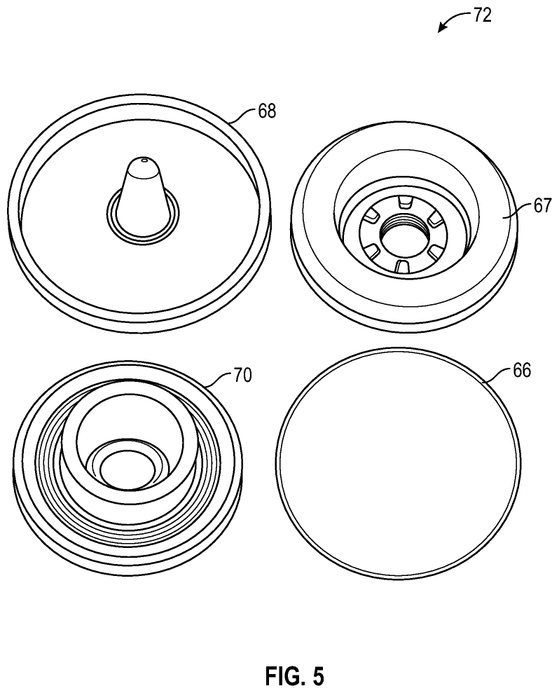

FIG. 5 shows an exploded snap kit 72 showing a disassembled snap 22. The sleeve snaps 56 and the anterior snaps 22 are size 20 snaps having a 1.2 inch diameter for ease of use by a user. The sleeve snaps 56 and the anterior snaps 22 are formed from a cap 66, a socket 67 and 70 and a stud 68 fabricated from a non-metallic material resistant to microbial contamination. The sleeve snaps 68, the snaps 22 and snaps 56, in an alternate embodiment are buttons.

The anterior snaps 22 provide easy access to visualize and treat wounds on the anterior of a user, such as surgical incisions on the chest or abdomen of a user.

The sleeve snaps 56 and the anterior snaps 22 are fabricated from a thermoplastic polymer selected from the group consisting of polypropylene, polyethylene, polyvinyl chloride, acrylonitrile butadiene styrene, and polycarbonate. Preferably, the snaps 68, 22, and 56 are radiolucent so that they can be used in an x-ray machine safely and have Hounsfield values of less than 60 HU and preferably less than 35.

Blood has a Hounsfield value of 30-35. In one embodiment the snaps have a Hounsfield value of less than 30 so that they do not interfere with the observation of blood in a diagnostic setting. The sleeve snaps 56 are spaced to enable an intravenous delivery tube to readily extend through the sleeves and into the arm of a user.

Carbon Enhanced Thermoplastic Materials

Thermoplastic materials are typically radiolucent, non-conductive and magnetically inert. However, strength and durability are often drawbacks of typical thermoplastic materials.

Medical use gowns are laundered very often. This wears the gown and its components rapidly. The sleeve snaps 56 and the anterior snaps 22 formed from a cap 66, a socket 67 and 70 and a stud 68 which are all fabricated from a thermoplastic polymer composite including carbon fibers. This composite structure yields increased strength and durability so that the sleeve snaps 56 and the anterior snaps 22 will not degrade with laundering, sterilization, daily use, and direct impact. Preferably a 10-40% carbon fiber content by volume in the thermoplastic material forms a suitable structure for the snaps 22 and 56 for the present invention. This carbon fiber content can be within the range of 50-65% in various alternate embodiments of the present invention.

The structural properties of thermoplastics snaps can be significantly enhanced by the addition of carbon fiber. The carbon fiber type, length, quantity, and orientation are a few of the important variables related to fiber selection. The present invention includes various embodiments of carbon fiber form and alignment in the snaps used for the present invention.

In one embodiment, the carbon fibers are specially treated to enhance bonding to the polymer matrix. This bonding is sometimes necessary to maximize the mechanical characteristics of the overall composite.

Fibers can be characterized by length (L) and diameter (D). This ratio is an aspect ratio and can be expressed as L/D. Including carbon fiber having a relatively higher aspect ratio may improve mechanical properties of the thermoplastics. Higher aspect ratios can be achieved by increasing the length or reducing the diameter of the fiber. For structural components, fiber length is frequently the focus for enhancing mechanical properties, and can vary from chopped to continuous.

Preferably, the snap is manufactured via injection molding from a polymer containing chopped carbon fibers in a thermoplastic resin. The percentage of chopped carbon fibers between 10 to 40% by volume.

In an alternate embodiment, continuous-fiber reinforcement improves strength and stiffness. The percentage of continuous fibers used is typically in the 50-65% range by volume in the thermoplastic resin. In one embodiment, the continuous-fiber orientations are unidirectional and bidirectional. In another embodiment, the continuous-fiber orientations are helical with the center of the snap being the axis. In yet another embodiment, the continuous-fiber orientations are in concentric rings around the axis of the snap.

In various embodiments of the invention, the snap is manufactured by one of the following methods: compression molding, injection molding, and extrusion. Preferably, the snaps and all components thereof are injection molded. The thermoplastic material has 10-40% chopped carbon fibers by volume in the thermoplastic material having an aspect ratio L/W of between 3/1 to 120/1. The thermoplastic material used for the snaps, in one embodiment of the invention the carbon fiber used has an aspect ratio L/D of 100 with a length of carbon fiber being on the average 0.6-0.8 mm (preferably 0.72 mm) and a width of 0.006 to 0.008 mm (preferably 0.072 mm). In another embodiment, the thermoplastic material has the mechanical characteristics expressed in the ranges of Table 2:

TABLE 2

| | |
|---|---|
| Characteristic | Range |

| | | |
|---|---|---|
| Flexural strength (ksi) | 1.37 | 67 |
| Flexural modulus (msi) | 7.7 | 10.5 |
| Specific strength | 2446 | 670 |
| (strength/density) | | |
| Specific modulus | 137.5 | 105 |
| (modulus/density) | | |
| Thermal expansion, | 1.6 × 10⁻⁶ | 14 × 10⁻⁶ |
| 70-300° F. (in./in./° F.) | | |
| Melting point (° F.) | 640 | 936 |

While various embodiments of the present invention are disclosed herein, these are provided by way of example only. The scope of the invention is set forth in the Appended claims.

What is claimed is:

1. A gown capable of use in a magnetic resonance imaging (MRI) scanner by a user, comprising:
an anterior panel having a length and two overlapping fabric sections join-able by a plurality of radiolucent anterior snaps that are alignable along a sagittal plane of the user when in use;
a posterior panel having a top defining a “v” shaped opening with a top tie integrated with the top of the “v” shaped opening on the top of the posterior panel, the “v” shaped opening having a center-line that is alignable along the sagittal plane of the user when in use;
two sleeves attached between the front panel and the rear panel and intersecting a coronal plane of the user when in use; and
a plurality of radiolucent sleeve snaps laterally aligned along each of the two sleeves and intersecting the coronal plane of the user when in use;
the sleeve snaps and the anterior snaps are manufactured from a thermoplastic material having a 10-40% carbon fiber content;
the sleeve snaps cooperate with the tie to enable selective and efficient access to the user during medical diagnosis and treatment without interfering with the magnetic resonance imaging scanner.

2. The gown as set forth in claim 1 further comprising:
two pockets formed on each side of the sagittal plane of the anterior panel, the fabric sections of each anterior panel defines a hole for enabling a telemetry wire to pass through each anterior panel, and each pocket overlaps the respective hole to normally shield the hole from view, and to enable the pocket to be capable of loosely holding the telemetry wire.

3. The gown as set forth in claim 2, wherein the anterior panel defines an anterior panel “v” neck when the plurality of radiolucent snaps are engaged during use, the “v” neck has an opening angle of between 45-65 degrees.

4. The gown as set forth in claim 3, wherein the “v” neck has an opening angle of approximately 55 degrees when the radiolucent snaps are snapped.

5. The gown as set forth in claim 3 further comprising a single side tie formed below the top tie to enable the posterior panel to overlap itself during use, the side tie connects one side of the posterior panel to the anterior panel.

6. The gown as set forth in claim 5, wherein the gown has a bottom edge, there are twelve anterior snaps, the first of which defines the base of the “v” neck opening angle of the anterior panel, the second of which is 6.3 inches below the first, the third is 8.2 inches below the second, the fourth is 8.2 inches below the third, the fourth is 8.2 inches below the third, the fifth is 8.8 inches below the fourth, the sixth is 8.2 inches below the fifth, the seventh is 8.2 inches below the sixth, the eighth is 7.9 inches below the seventh, the ninth is 7.9 inches below the eighth, the tenth is 8.2 inches below the ninth, the eleventh is 8.0 inches below the tenth, the twelfth is 7.9 inches below the eleventh, and the distance from the twelfth to the bottom edge of the gown is 3.8 inches, whereby the distance between the snaps decreases near the bottom edge to assure that the anterior panel will remain aligned when the user walks.

7. The gown as set forth in claim 6, wherein each of the two sleeves has four sleeve snaps.

8. The gown as set forth in claim 7, wherein each of the sleeve snaps are size 20 snaps having a 1.2 inch diameter for ease of use by a user.

9. The gown as set forth in claim 1, wherein the sleeve snaps and the anterior snaps are formed from a cap, a socket and a stud fabricated from a non-metallic material resistant to microbial contamination.

10. The gown as set forth in claim 9, wherein the sleeve snaps and the anterior snaps are fabricated from a thermoplastic polymer selected from the group consisting of polypropylene, polyethylene, polyvinyl chloride, acrylonitrile butadiene styrene, and polycarbonate.

11. The gown as set forth in claim 10, wherein the sleeve snaps and the anterior snaps are powder coated to inhibit microbial contamination.

12. The gown as set forth in claim 10, wherein the sleeve snaps are fabricated from injection molding and are impregnated with an anti-microbial compound.

13. A radiolucent gown for selective use with magnetic resonance imaging scanner and an electrocardiogram device on a user, comprising:
an anterior panel having a length and two overlapping fabric sections join-able by a plurality of non-metallic anterior snaps that are alignable along a sagittal plane of the user when in use;
a posterior panel having a top defining a “v” shaped opening with a top tie integrated with the top of the “v” shaped opening on the top of the posterior panel, the “v” shaped opening having a center-line that is alignable along the sagittal plane of the user when in use;
two sleeves attached between the front panel and the rear panel and intersecting a coronal plane of the user when in use;
the sleeves have a plurality of non-metallic sleeve snaps and the method further includes snapping the sleeve straps to secure the gown to the user;
the sleeve snaps and the anterior snaps are radiolucent and being manufactured from a thermoplastic material having a 10-40% carbon fiber content;
the sleeve snaps laterally align along each of the two sleeves and intersecting the coronal plane of the user when in use; the sleeve snaps and the anterior snaps cooperate with the tie to enable selective and efficient access to the user during medical diagnosis and treatment without interfering with the magnetic resonance imaging scanner;
two pockets formed on each side of the sagittal plane of the anterior panel, the fabric sections of each anterior panel defines a hole for enabling a telemetry wire to pass through each anterior panel, and each pocket overlaps the respective hole to normally shield the hole from view, and to enable the pocket to be capable of loosely holding the telemetry wire.

14. A radiolucent gown, comprising:
fabric formed into an open anterior panel, a posterior panel and sleeves;
radiolucent anterior snaps for selectively securing the anterior panel;
radiolucent sleeve snaps arranged along a length of each sleeve; and
the radiolucent anterior snaps and the radiolucent sleeve snaps are manufactured from a composite material having a 10-45% chopped carbon fiber content,
wherein the carbon fibers have an average aspect ratio (L/W) of between 50-120.

15. The gown as set forth in claim 14, wherein the radiolucent sleeve snaps and the radiolucent anterior snaps have a radio density of less than 60 Hounsfield units.

16. The gown as set forth in claim 14, wherein radiolucent sleeve snaps and the radiolucent anterior snaps have a radio density of less than 35 Hounsfield units.

17. The gown as set forth in claim 14, wherein radiolucent sleeve snaps and the radiolucent anterior snaps have a radio density of between 35-60 Hounsfield units.

18. The gown as set forth in claim 17, wherein the radiolucent sleeve snaps and the radiolucent anterior snaps are manufactured by injection molding.

19. The gown as set forth in claim 17, wherein the radiolucent sleeve snaps and the radiolucent anterior snaps are the same shape and size.

20. The gown as set forth in claim 19, wherein each of the radiolucent sleeve snaps and the radiolucent anterior snaps are size 20 snaps, having a 1.2 inch diameter.
